(19) United States
(12) Patent Application Publication
(10) Pub. No.: US 2021/0379227 A1
(43) Pub. Date: Dec. 9, 2021

(54) APPARATUS AND METHOD FOR OPENING AND CLOSING A MEDIA EXCHANGE OPENING BY MEANS OF AN ELECTROMECHANICAL ACTUATOR

(71) Applicant: Aesculap AG, Tuttlingen (DE)

(72) Inventor: Matthias Schweizer, Tuttlingen (DE)

(21) Appl. No.: 17/284,013

(22) PCT Filed: Oct. 10, 2019
(86) PCT No.: PCT/EP2019/077512

§ 371 Date: Apr. 9, 2021

(30) Foreign Application Priority Data
Oct. 12, 2018 (DE) 10 2018 125 314.1

(51) Int. Cl. A61L 2/26

(52) U.S. Cl. CPC A61L 2/26; A61L 2202/14; A61L 2202/182; A61L 2202/121; A61L 2202/24; A61L 2202/122

(57) ABSTRACT

A sterilization container includes a housing having at least one opening and at least one valve unit for opening and closing the opening. The valve unit is of the electromechanical type having an electromechanical actuator which is controllable or actuated by a control unit. Furthermore, the valve unit has a valve body to which the control unit is coupled, wherein the opening can be opened and can be closed by the valve body, more precisely the actuator.

### CROSS-REFERENCE TO RELATED APPLICATION(S)

This application is the United States national phase entry of International Application No. PCT/EP2019/077512, filed Oct. 10, 2019, and claims the benefit of priority of German Application No. 10 2018 125 314.1, filed Oct. 12, 2018. The contents of International Application No. PCT/EP2019/077512 and German Application No. 10 2018 125 314.1 are incorporated by reference herein in their entireties.

### FIELD

The present disclosure relates to a sterilization container having at least one opening in its housing and at least one valve unit opening and closing the opening, a sterilization system, and a method for opening and closing the opening by means of a valve unit.

### BACKGROUND

Medical sterilization containers (also sterile containers, sterilization cases or sterile cases) are used to sterilize, transport and store surgical instruments or material. An opening is required so that hot steam can enter the medical sterilization container during the sterilization process in a sterilizer, preferably in an autoclave. However, in order to prevent germs, bacteria or the like from entering the container after sterilization, the opening is fitted with a sterilization filter prior to the sterilization process. The sterilization filter allows fluid exchange, but does not allow unwanted pathogens to enter the medical sterilization container. In other words, the opening of the medical sterilization container is closed by a filter unit that has many small fluid exchange openings/pores that allow exchange of fluids, or molecules thereof, but prevents the entry of germs, bacteria or the like that are larger than the molecules of the fluids after the sterilization process.

Sterile filters for sterile filtration for medical sterilization containers are usually designed in the form of a flat circular filter disc. The filter disc has fluid exchange openings (also called pores) which allow the exchange of fluids, in particular gases, liquids or gas-liquid mixtures, between the environment and the interior of a medical sterilization container. The medical sterilization filter is thereby held in a filter holder attached to the medical sterilization container.

In this regard, the filter holder clamps the sterilization filter between a first holding surface and a second holding surface.

In sterile filtration, microorganisms are separated from a fluid by filtration. Membranes with a pore diameter of 0.22 μm are usually used as filters. However, it can also be practical to use smaller pore diameters, such as 0.1 μm. This allows only small molecules to pass through the membrane, while larger particles such as bacteria are retained. Sterile filtration is often used to sterilize heat-sensitive solutions, such as tissue culture solutions containing serum. Other main applications of sterile filtration, besides the already known fields of application, include the filtration of aqueous solutions, heat-sensitive nutrient solutions, vitamin solutions, sera, viral vaccines, plasma fractions and protein solutions.

Sterilization requires an exchange of media between the environment and the inside of a sterile container in order to bring the sterilization medium (for example steam, H₂O₂ gas, etc.) into the container and thus to the goods to be sterilized. Small pressure differences, which can occur during storage and transport, are compensated by various media-permeable filter systems. In particular, various filter media such as single-use filters made of known filter materials or permanent filters made of PTFE are used. Also known is a system available from the company KLS Martin under the trademark MICROSTOP®, which works via a pasteuric loop.

Larger pressure differences, such as those occurring during the pressure change phases in the sterilizer, cannot be compensated by these systems, since their media permeability is too low for this. Therefore, (hydraulically/pneumatically controlled) valves or valve-like mechanisms are used in the sterilizer for rapid compensation of positive and negative pressures inside the container. These create a larger opening gap for rapid ventilation/venting of the interior space of the sterile container.

In the prior art, sterile containers use a so-called flexible filter holder for this purpose, which bends elastically in the event of negative pressure in the container and creates a gap-like bypass. In the event of overpressure in the container, the complete lid lifts off via a spring-loaded closure. Furthermore, special valve systems are used in the prior art, including, inter alia, double-acting valve systems, thermally activated valve systems and absolute-pressure activated valve systems. The latter are used in particular for condensate drainage, but also function by creating a bypass through which the condensate drains.

The disadvantage of previous systems is that passive valve systems are used. These are usually spring-loaded or operate by means of elastic deformation of valve components. The current valves operate depending on the prevailing differential pressure between the sterilization chamber in the sterilizer and the container interior, temperature or absolute pressure. The systems are therefore reactive and inert. Due to the purely mechanical implementation of the valves, they are also highly tolerance-dependent and fluctuate in the opening pressures. Separate systems for negative pressure and positive pressure are also sometimes required. Misopenings of the valve systems cannot be ruled out, for example in the case of damaged systems. Temperature-loaded systems can open, for example, due to unintentional exposure to heat.

Thus, the object of the invention is to provide an active system for opening and closing media exchange openings in sterile containers and to eliminate the preceding disadvantages. In other words, the object is to reduce or eliminate the opening tolerances and closing tolerances of the media exchange system. Furthermore, false openings of the media exchange system shall be avoided (e.g. in case of damaged systems). In addition, the valve position is to be monitored, both before and after the sterilization process, thus providing an automated check of the system's functionality. A further object is to optimize the sterilization process and to simplify the media exchange.

### SUMMARY

A sterilization container according to a first aspect of the present invention comprises a housing having at least one opening and at least one valve unit opening and closing the opening. The valve unit is of electromechanical design and comprises an electromechanical actuator. Furthermore, the sterilization container comprises a control unit. The electromechanical actuator can be regulated/controlled by the control unit or can be driven by it. The valve unit further comprises a valve body, for example in the form of a sterile filter unit, which is coupled to the electromechanical actuator. The opening can be opened and/or closed by the actuator via the valve body.

In other words, the sterilization container according to the invention has an opening/media exchange opening/ventilation opening. This opening can be opened and/or closed by the valve unit. The valve unit is of electromechanical design, which means that the valve unit has elements that are electrically driven, for example an electromechanical actuator. Since the valve unit is of the electromechanical type, the sterilization container comprises at least one power source, such as batteries, accumulators, capacitors or the like. Furthermore, the sterilization container has a control unit/control device/control unit that drives the electromechanical valve unit, whereby the actuator moves a valve body for opening and/or closing the opening.

The valve unit is a component for shutting off or controlling/regulating the flow of fluids. For this purpose, the valve unit has the valve body/closure part/shut-off element (e.g. plate, cone, ball or needle), which is preferably moved approximately parallel to the direction of flow of the fluid. The flow is interrupted by pressing the closing part with a sealing surface against a suitably shaped opening, i.e. the valve seat or seal seat.

An electromechanical valve unit is understood to be a valve unit in which mechanical processes/valve operations are generated with the aid of electrical drives.

Actuators or actuating elements convert electrical signals (e.g. commands issued by the control unit/control computer) into mechanical motion or other physical quantities and thus actively intervene in the opening/closing process. A further aspect of the invention, which may be considered independently, is that the electromechanical actuator may be an electric motor, a linear motor, a piezomotor or the like. Other types of actuators may also be used, such as a hydraulic actuator, a pneumatic actuator, or a magnetosensitive actuator.

A further aspect of the invention, which may be considered independently, is that the displacement or force transmission of the actuator may be effected by known mechanical principles, such as by a gear transmission, by the stroke of a thread/fine thread, by a lever mechanism, by a magnetic mechanism, or the like. Preferably, the stroke is linear.

A further aspect of the invention, which may be considered independently, is that the valve body is a sterilization filter unit or that the valve body is a lid of the sterilization container. In other words, the valve body can be used for any opening of the sterile container. In this way, the actuator can move a portion of the valve unit or the valve unit as a whole to open or close an opening. For example, the actuator can lift/move/tilt a lid or the like of the sterile container, thus allowing fluid exchange between the interior of the container and the exterior of the container. However, the actuator may also move a part of the sterile filter unit, such as a sterile filter, in a holder provided for this purpose, thereby uncovering or closing an opening. However, it is also possible to open or close an opening specially provided and adapted for a media exchange by means of the valve unit and thus by means of the actuator moving a valve body. Furthermore, a combination of the aforementioned possibilities can be realized.

A further aspect of the invention, which may be considered independently, is that the sterile container preferably comprises at least one sensor unit provided and adapted to communicate with the control unit. In other words, the sterilization container comprises a sensor which is in contact with the control unit and/or the actuator such that the control unit receives a signal from the sensor and the actuator is driven/actuated accordingly. In yet other words, a signal from the sensor causes the opening to be closed, opened, or optionally to be opened to a predetermined degree/opening state. The sensor in the sterile container may be a pressure sensor and/or a temperature sensor and/or a humidity sensor. The pressure sensor preferably measures a pressure difference between the internal pressure of the housing and the ambient pressure. The sensor thus detects at least one pressure difference between the sterile container interior and the sterilizer chamber. According to the pressure difference, the valve system/valve unit is driven based on a defined limit value and the media exchange opening is released/opened. Analogously, what is stated above can also be applied to other types of sensors. The temperature sensor preferably measures an outside temperature and/or an inside temperature of the sterile container. The humidity sensor preferably measures an external humidity and/or an internal humidity of the sterile container. However, the sensor unit can also comprise a combination of different sensors in particular according to the preceding list.

In a sensor-based system, the actuator is controlled, preferably actively, on the basis of the sensor data. The sensor unit can be mounted inside the sterile container, on the outside of the sterile container or on the outside and inside. An embodiment is also possible where the sensor is mounted in the sterilizer or its chamber and sends the sensor signals to a receiver unit in or on the sterilization container. Sensor modules are also conceivable, which can be added to a sieve basket, a container, a soft package or a wrap-packaged sterile material.

A further aspect of the invention, which may be considered independently, is that the valve unit preferably comprises at least one transmitter and/or receiver, each of which is provided and adapted to communicate with the control unit. In other words, the sterilization container preferably comprises a receiver that receives signals from a transmitter in the sterilizer or its chamber and actuates the actuator when sterilization starts, thus opening the opening, or closes the opening when sterilization ends. Thus, the sterile container preferably has the receiver, whereby when a signal is received indicating the start of a process, the opening can be opened, and when a signal is received indicating the end of a sterilization process, the opening can be closed. The received signal can alternatively also be received by the transmitter of a transmitter unit (analogous to the transmitter unit disclosed above) in the sterilizer. In an alternative embodiment, the sterile container has a transmitter so that sterilization only takes place when the sterile container has been in the sterilizer for a predetermined time (e.g. 10 seconds). Preferably, the transmitter and/or the receiver can also be an RFID chip, which furthermore also stores data about contents, sterilization processes, etc. and is thus tracked in order to follow the process sequence of the sterilization container live/online.

In yet other words, the actuator is driven as a function of the sterilization process and/or the prevailing ambient conditions. In a process-controlled media exchange system, the actuator is driven by active communication between the sterilizer and the actuator controller. The actuator is driven according to the respective, current process step and independent of the differential pressure, temperature and/or absolute pressure, thus opening or closing the valve. Thus, the opening is opened by the actuator at the beginning of the sterilization process, or as soon as the sterile container is in the sterile area, and closed by the actuator before or at the end of the sterilization process, i.e. before the sterile container leaves the sterile area.

Opening and closing of the media exchange opening thus takes place depending on the prevailing ambient conditions and/or depending on the sterilization process, depending on whether the sterilization container has a sensor and/or transmitter and/or receiver. As soon as the media exchange opening is open, a direct exchange of media takes place between the sterilizer and the sterilization container interior. In other words, sterilization of the sterile container interior takes place when the media exchange opening is open. Both systems, i.e. the actuator module and the filter unit, can be designed as a combined system or can also be installed as separate systems or units. In yet other words, the valve unit may be designed to include the actuator, optionally the control unit, optionally the power supply, the valve body, and optionally a transmitter and/or a receiver, and optionally the sensor. In yet other words, a separate valve unit comprising all of the preceding elements can be inserted into an opening of the sterile container.

A further aspect of the invention relates to a method to control opening and closing of an opening in a sterile container. The sterile container comprises at least one control unit, at least one electromechanical actuator, at least one power supply, at least one valve unit with preferably at least one valve body and at least one signal generator. The signal generator is preferably a sensor and/or transmitter and/or receiver. The method comprises the following steps in the order indicated:
Receiving a first signal of the signal generator from the control unit,
actuating the actuator by a signal from the control unit which has generated the control signal based on the received first signal,
opening the opening by means of a movement of the valve body by the actuator,
receiving a second signal of the signal generator, by the control unit,
actuating the actuator by a control signal from the control unit, which generated the control signal based on the received second signal, and
closing the opening by means of a movement of the valve body by the actuator.

In other words, the control unit receives a first signal at the start of a sterilization from a sensor and/or transmitter and/or receiver, which causes an actuator to be actuated. The signal from the sensor and/or transmitter and/or receiver can first be passed to a control unit, which in turn causes the actuator to be actuated, but the signal can also go directly to the actuator. The actuator opens the opening by moving the valve body so that it releases or opens the opening. At the end of the sterilization, the control unit receives a second signal from a sensor and/or transmitter and/or receiver, which actuates the actuator. The signal from the sensor and/or transmitter and/or receiver can first be passed to a control unit, which in turn causes the actuator to be actuated, but the signal can also go directly to the actuator. The actuator closes the opening by moving the valve body so that it closes the opening.

In summary, the invention relates to a sterilization container having a housing with at least one opening and at least one valve unit opening and closing the opening. The valve unit is of electromechanical design with an electromechanical actuator which can be controlled or driven by a control unit. Furthermore, the valve unit has a valve body which is coupled to the control unit, wherein the opening can be opened and closed via the valve body, more precisely via the actuator. The sterile container thus allows an active exchange of media. The active media exchange is achieved by the electromechanical actuator. Active control of the media exchange in the sterilization process is thus made possible. The active control is achieved by the control unit (preferably a CPU). A sensor data-controlled system, which is dependent on the pressure difference, for example, and a process-controlled system, which opens and closes the opening depending on the process phase, are thus provided. Active communication between the sterilizer and the actuator forms the basis of the process-controlled system, and active communication between the sensor unit and the actuator forms the basis of the sensor-data controlled system. In this context, the actuator and the sensor unit and/or the control unit can be designed as an independent module.

### BRIEF DESCRIPTION OF THE DRAWING FIGURES

The invention is explained in more detail below by means of a preferred configuration example with reference to the accompanying figures.

FIG. 1 shows a schematic representation of a sterile container, and

FIG. 2 shows a schematic representation of an inner side of a side wall of the sterile container.

### DETAILED DESCRIPTION

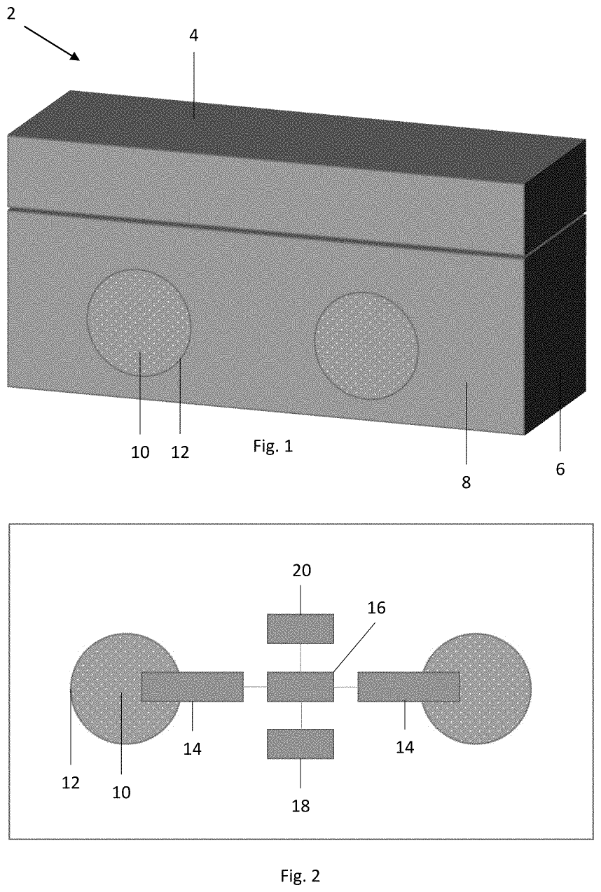

FIG. 1 shows the schematic representation of a sterile container 2 with a lid 4 and a container tray 6. The lid 4 closes the sterile container 2 by being placed on the container tray 6 in a sealing manner. The body 6 has a side wall 8 in which preferably two sterile filter units 10 are inserted in preferably two openings 12 formed in the side wall 8.

FIG. 2 schematically shows the inside of the side wall 8 of the housing tray 6. A respective actuator 14, for example in the form of a piezo drive, is connected to a respective sterile filter unit 10. Each sterile filter unit 10 connected to the actuator 14 thus forms a valve body which is preferably movably seated on the respective opening 12 and can thus be moved back and forth between at least two relative positions to the opening 12, namely a closed position in which the sterile unit is thus firmly seated on the opening 12 and an open position in which the sterile filter unit 10 is spaced apart from the opening thus forming a gap.

The actuator 14 can move the sterile filter unit 10 and can thus open or close the opening 12. The actuator 14 is controlled by a control unit 16. The control unit 16 in turn receives a signal from a signal generator 18, wherein the signal generator 18 is a sensor and/or transmitter and/or receiver. The signal represents, for example, a pressure, a temperature and/or a humidity inside and/or outside the container 2.

The control unit 16 as well as the actuator 14 and the signal generator 18 are connected to a power supply 20. Alternatively, the signal generator can also be a passive sensor that is not directly connected to the power supply (battery) 20. As can be seen from FIGS. 1 and 2, the valve unit constructed in this way (including actuator, control unit and battery) is located inside the container 2 and is thus protected from damage by the container wall 8.

1. A sterilization container comprising a housing with an opening and at least one valve unit for opening and closing the opening, wherein the valve unit is of electromechanical design, with an electromechanical actuator which is controllable or driven by a control unit, and is coupled to a valve body by means of which the opening can be opened and closed by the actuator.

2. The sterilization container according to claim 1, wherein the valve body is a sterilization filter unit.

3. The sterilization container according to claim 1, wherein the valve body is a lid of the sterilization container which can be placed in a sealing manner on a container tray.

4. The sterilization container according to claim 1, wherein the sterilization container comprises a sensor unit which is provided and adapted to communicate with the control unit.

5. The sterilization container according to claim 1, wherein the sensor unit has or is at least one pressure sensor.

6. The sterilization container according to claim 1, wherein the sensor unit has or is at least one temperature sensor.

7. The sterilization container according to claim 1, wherein the sensor unit has or is at least one humidity sensor.

8. The sterilization container according to claim 1, further comprising at least one transmitter and/or receiver, each provided and adapted to communicate with the control unit.

9. A sterilization system comprising a sterilizer and at least one sterilization container according to claim 1.

10. The sterilization system according to claim 9, wherein the sterilizer is provided with at least one receiver and/or transmitter provided and adapted to communicate with a transmitter and/or receiver of the sterile container.

11. A control method for opening and closing an opening in a sterile container comprising a control unit, an electromechanical actuator, a power supply, a valve body and a signal generator, comprising the following steps:
receiving a first signal of the signal generator, by the control unit;
actuating the actuator by an actuation signal from the control unit, in response to the first signal;
opening the opening by means of a movement of the valve body by the actuator in response to the second signal, and;
receiving a second signal of the signal generator by the control unit;
actuating the actuator by an actuation signal by the control unit in response to the second signal; and
closing the opening by movement of the valve body by the actuator in response to the actuation signal.
